(19) United States
(12) Patent Application Publication
(10) Pub. No.: US 2025/0198550 A1
(43) Pub. Date: Jun. 19, 2025

(54) Negative Pressure Connector Seal

(71) Applicant: C. R. Bard, Inc., Franklin Lakes, NJ (US)

(72) Inventors: Gabriel A. Johnston, Broomfield, CO (US); Sean E. Walker, Platteville, CO (US); Madeline Stich, Thornton, CO (US); Brett R. Skelton, Louisville, CO (US); Karthik Ganesan, Broomfield, CO (US)

(21) Appl. No.: 19/068,969

(22) Filed: Mar. 3, 2025

Related U.S. Application Data
(63) Continuation of application No. 17/848,074, filed on Jun. 23, 2022, now Pat. No. 12,241,570.
(60) Provisional application No. 63/219,247, filed on Jul. 7, 2021.

(51) Int. Cl. F16L 21/00; A61F 7/00; F16K 15/14

(52) U.S. Cl. CPC F16L 21/005; A61F 7/0085; F16K 15/1471

(57) ABSTRACT

A fluid connector system includes a first connector adjoining a second connector at a junction, and a sealing member coupled to the first connector and the second connector around the junction. The sealing member can include an annular wall having a first annular portion configured to engage the first connector and a second annular portion configured to engage the second connector. The sealing member can include an annular chamber extending between the first annular portion and the second annular portion. The annular chamber can be formed by the first annular portion sealing against an outer surface of the first connector and the second annular portion sealing against an outer surface of the second connector when a pressure between the sealing member and the first connector and the second connector is negative.

### PRIORITY

This application is a continuation of U.S. patent application Ser. No. 17/848,074, filed Jun. 23, 2022, now U.S. Pat. No. 12,241,570, which claims the benefit of priority to U.S. Provisional Application No. 63/219,247, filed Jul. 7, 2021, each of which is incorporated by reference in its entirety into this application.

### BACKGROUND

Targeted temperature management (TTM) systems circulate a fluid (e.g., water) between one or more thermal contact pads coupled to a patient and a TTM fluid control module via a fluid deliver line (FDL). Fluid connectors between the FDL and the pads and between the FDL and the control module provide for the selective connecting and disconnecting of the components. As a clinician may connect and disconnect the components for each TTM procedure (potentially several times per day), it is desirable for the connection to be simple, straight forward, and require minimal forces. It is also important for the fluid seal between the connectors to be reliable.

To prevent water leakage from the TTM system, the system may be configured to operate under a negative pressure so that, in the case of a leaking connection, air may leak into the system as opposed as to water leaking from the system. Disclosed herein are embodiments of devices and methods for utilizing negative internal pressure to improve connector seal reliability while minimizing clinician applied forces to make the connection.

### SUMMARY OF THE INVENTION

Briefly summarized, disclosed herein is a sealing member for providing a seal between fluid connectors. The sealing member includes a tubular member defining a lumen extending between a first end and a second end. The tubular member is configured to receive a first connector via the first end and a second connector via the second end. The tubular member includes an annular wall extending between a first annular portion adjacent the first end and a second annular portion adjacent the second end. The first annular portion is configured to engage the first connector and the second annular portion is configured to engage the second connector. When a pressure within the lumen is negative, atmospheric acting inward on the annular wall compresses the sealing member to define a contact force between the second annular portion and the second connector sufficient to define a fluid seal between the sealing member and the second connector.

The pressure within the lumen is defined in response to a fluid pressure within a lumen extending through the first and second connectors and the lumen may be in fluid communication with the lumen extending through the first and second connectors.

In some embodiments, when the pressure within the lumen is negative, atmospheric acting on the annular wall compresses the sealing member to define a contact force between the first annular portion and the first connector sufficient to define a fluid seal between the sealing member and the first connector.

In some embodiments, the first annular portion is attached to the first connector and may also be sealably attached to the first connector. The second annular portion is configured to slidably engage the second connector when a non-negative pressure is defined within the lumen.

One of the first connector or the second connector may be attached to one of a fluid delivery line or a thermal contact pad of a targeted temperature management system, and the other one of the first connector or the second connector may be attached to the other one of the fluid delivery line or a thermal contact pad. The fluid pressure may be defined in accordance with operation of the targeted temperature management system.

The sealing member may further include a septum extending across the lumen between the first connector and the second connector, and the septum includes a pressure actuated valve. The valve is configured to (i) prevent fluid flow through the septum when a fluid pressure across the septum is below a defined pressure limit, and (ii) allow fluid flow through the septum when the fluid pressure across the septum exceeds the defined pressure limit.

Also disclosed herein is a fluid connector system, including a first connector, a complementary second connector fluidly coupled with the first connector, and a tubular sealing member defining a lumen extending between a first end and a second end. The tubular member is configured to receive the first connector via the first end and the second connector via the second end. The tubular member includes an annular wall extending between a first annular portion adjacent the first end and a second annular portion adjacent the second end. The first annular portion is configured to engage the first connector and the second annular portion is configured to engage the second connector. When a pressure within the lumen is negative, atmospheric acting on the annular wall compresses the sealing member to define a contact force between the second annular portion and the second connector sufficient to define a fluid seal between the sealing member and the second connector.

The pressure within the lumen is defined in response to a fluid pressure within a lumen extending through the first and second connectors and the lumen may be in fluid communication with the lumen extending through the first and second connectors.

In some embodiments, when the pressure within the lumen is negative, atmospheric acting on the annular wall compresses the sealing member to define a contact force between the first annular portion and the first connector sufficient to define a fluid seal between the sealing member and the first connector.

In some embodiments, the first annular portion is attached to the first connector and may also be sealably attached to the first connector. The second annular portion is configured to slidably engage the second connector when a non-negative pressure is defined within the lumen.

In some embodiments, the second connector includes a first annular connector wall and a second annular connector wall spaced radially outward of the first annular connector wall. A lateral bottom wall extends between the first and second annular connector walls to define an annular cavity, and the second annular portion is disposed within the annular cavity.

An inside surface of the second annular portion is configured to slidably engage the first annular connector wall, and an outside surface of the second annular portion is configured to slidably and sealably engage the second annular connector wall. When a negative pressure is defined within the lumen, a fluid seal is defined between the inside surface of the second annular portion and the first annular connector wall, and when a non-negative pressure is defined within the lumen, a fluid seal is defined between the outside surface of the second annular portion and the second annular connector wall.

One of the first connector or the second connector may be attached to a component of a targeted temperature management (TTM) system, and the component may be one of a TTM module, a fluid delivery line, or thermal contact pad. The fluid pressure may be defined in accordance with operation of the targeted temperature management system.

The sealing member may include a septum extending across the lumen between the first connector and the second connector, and the septum includes a pressure actuated valve. The valve configured to prevent fluid flow through the septum when a fluid pressure across the septum is below a defined pressure limit, and allow fluid flow through the septum when the fluid pressure across the septum exceeds the defined pressure limit.

Also disclosed herein is a method of defining a seal between fluid connectors. The method includes (i) providing a tubular sealing member defining a lumen extending between a first end and a second end, (ii) inserting a first connector into the sealing member via the first end to define a first engagement, (iii) inserting a second connector into the sealing member via the second end to define a second engagement, (iv) establishing a negative pressure within the lumen to transition the sealing member from a first engagement configuration to a second engagement configuration. In the first engagement configuration, at least one of the first connector or the second connector is not sealably coupled with the sealing member, and in the second engagement configuration, atmospheric pressure compresses the sealing member to define a fluid seal between the least one of the first connector or the second connector and the sealing member. In the second engagement configuration, separation of the second connector from the first connector may be prevented.

The method may further include (i) establishing a non-negative pressure within the lumen to transition the sealing member from the second engagement configuration to the first engagement configuration and (ii) separating the second connector from the first connector.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a side cross-sectional view of a sealing member for providing a fluid seal between connectors, in accordance with some embodiments.

FIG. 2 illustrates a side cross-sectional view of a connector system including a second embodiment of the sealing member, in accordance with some embodiments.

FIG. 3A illustrates a side cross-sectional view of a third embodiment of the sealing member, in accordance with some embodiments.

FIG. 3B illustrates a septum of the sealing member of FIG. 3A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

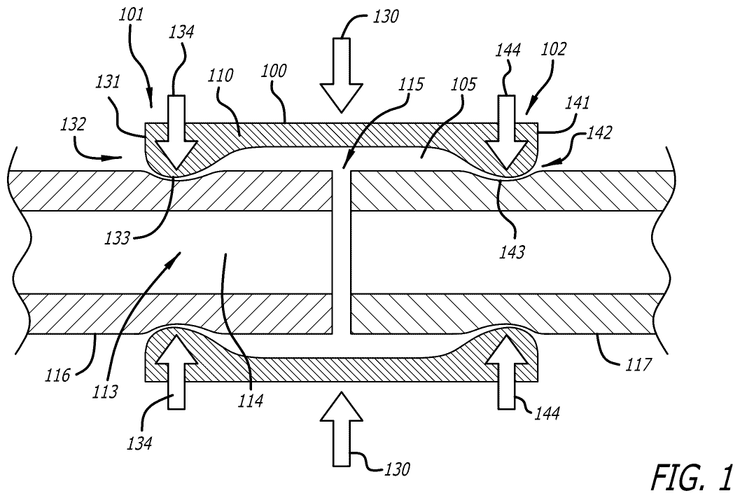

FIG. 1 illustrates a cross-sectional side view of a sealing member 100 coupled between representative fluid connectors, i.e., the first connector 116 and the second connect 117, in a connected state. A fluid lumen 113 extends longitudinally through the connectors 116, 117. The sealing member 100 is configured to provide a fluid seal between the first and second connectors 116, 117. In some embodiments, the sealing member 100 may define a primary seal between the connectors. In other embodiments, the sealing member 100 may define a secondary seal (i.e., a backup seal) between the first and second connectors 116, 117.

In some embodiments, the first and second connectors 116, 117 may be attached to components of a TTM system (not shown). For example, the first and second connectors 116, 117 may define a fluid connection between a fluid delivery line and a thermal pad. In another example, the first and second connectors 116, 117 may define a fluid connection between a fluid delivery line and a TTM module.

The sealing member 100 may generally define a tubular shape having an annular wall 110. The annular wall 110 may extend circumferentially around and longitudinally along each of the connectors 116, 117. The sealing member 100 may be positioned with respect to the connectors 116, 117 so that a junction point 115 of the connectors 116, 117 is located between a first end 101 and a second end 102 of the sealing member 100.

The sealing member 100 may be an elastic sleave/shroud configured to deflect or deform in response to externally applied forces. In some embodiments, the sealing member 100 may include one or more deflectable/deformable portions. The sealing member 100 or at least a portion thereof may be formed of a flexible/deformable material such as silicone, ethylene propylene diene monomer rubber (EPDM), a natural rubber, or any other suitably flexible material. In some embodiments, the sealing member 100 may include supporting structural elements, such as a coil, longitudinal stiffening wires, circular rings, or any other structure elements consistent with the functionality of the sealing member 100.

The sealing member 100 may be attached to the first connector 116 to inhibit or prevent longitudinal displacement of the sealing member 100 with respect to the first connector 116. In some embodiments, rotation of the sealing member 100 with respect to the first connector 116 may also be inhibited. The attachment of the sealing member 100 to the first connector 116 may define a fluid seal 132 between sealing member 100 and the first connector 116.

The sealing member 100 may be coupled with the first connector 116 via a contact force between the sealing member 100 and the first connector 116. For example, a first engagement portion 131 of the sealing member 100 may be sized to fit within a recess 133 of the first connector 116 while defining an interference fit with the first connector 116. In some embodiments, the sealing member 100 may include a separate device (e.g., a band clamp, not shown) to define the contact force. In other embodiments, the sealing member 100 may be bonded to the first connector 116 via an adhesive. The coupling of the first engagement portion 131 with the first connector 116 may define the fluid seal 132 between the sealing member 100 and the first connector 116. By way of summary, the sealing member 100 may be permanently attached to the first connector 116 or selectively attached to and/or detached from the first connector 116.

The sealing member 100 engages the second connector 117 via a second engagement portion 141. The second engagement portion 141 is sized to fit within a recess 143 defining an interference fit with the second connector 117. The interference fit is defined to accommodate longitudinal displacement of the second connector 117 relative to sealing member 100. In other words, a contact force 144 between the second engagement portion 141 and second connector 117 may be sufficiently minimal to allow the second connector 117 to be inserted into and extracted from the sealing member 100 manually be a clinician while also defining a seal 142 between the sealing member 100 and the second connector 117.

The sealing member 100 defines an annular chamber 105 (e.g., annular space or gap) between the connectors 116, 117 and the annular wall 110. The chamber 105 is bounded on the ends by the engagement portions 131, 141. The chamber 105 may be in fluid communication with the lumen 113 via a leak path between the connectors 116, 117 at the junction point 115. As such, the fluid pressure 114 within the lumen 113 may define a chamber pressure 106 of the chamber 105. In an instance of a negative pressure within the lumen 113, the resulting negative chamber pressure 106 causes atmospheric pressure to exert a radially inward force 130 on the annular wall 110. In such an instance, the radially inward force 130 causes an increase in the contact force 144 between the second engagement portion 141 and the second connector 117. The seal 142 between the second engagement portion 141 and the second connector 117 may be enhanced by the increase in the contact force 144 resulting from the negative chamber pressure 106.

In some embodiments, the radially inward force 130 applied to the annular wall 110 may also increase a contact force 134 between the first engagement portion 131 and the first connector 10. Consequently, the negative pressure 114 with the lumen 113 may define a greater integrity of the seal 132.

The sealing member 100 is configured to engage the second connector 117 according to a first engagement configuration and a second engagement configuration as defined by the pressure 114 within the lumen 113. More specifically, the sealing member 100 may be disposed in a first engagement configuration when the pressure 114 is non-negative. The sealing member 100 may transition toward the second engagement configuration in response to a negative pressure 114. In the first engagement configuration, the sealing member 100 may facilitate coupling and decoupling of the connectors 116, 117. As such, longitudinal and/or rotational displacement between the sealing member 100 and the second connector 117 is allowed in the first engagement configuration.

The second engagement configuration defines enhanced engagement properties over the first engagement configuration. The second engagement configuration defines a greater integrity of the seal 142 than the first engagement configuration. Similarly, the second engagement configuration may define a greater frictional force between the sealing member 100 and the second connector 117 resisting longitudinal and rotational displacement of the second connector 117 with respect to the sealing member 100.

In use, the sealing member 100 may automatically transition between the first engagement configuration and the second engagement configuration. In some embodiments, the sealing member 100 may transition from the second engagement configuration to the first engagement configuration to facilitate connection and/or disconnection of the connectors 116, 117. Similarly, the sealing member 100 may transition from the first engagement configuration to the second engagement configuration to establish the seal 142 only when the first connector 116 is coupled with the second connector 117. Additionally, the sealing member 100 may transition from the first engagement configuration to the second engagement configuration in response to a change the pressure 116 toward the negative and in some embodiments, the negative pressure may be defined by the TTM module.

A method of using the sealing member 100 may include the following steps or processes. The sealing member 100 is coupled with the first connector. The first connector 116 is coupled with the second connector 117 during which the second connector 117 is inserted within the sealing member 117. A negative pressure is established within the lumen 113. In the event of a leak between the connectors 116, 117, the negative causes the sealing member 100 to form a seal 142 between the sealing member 100 and the second connector 117. In some embodiments, the vacuum also causes the sealing member 100 to form a seal 132 between the sealing member 100 and the first connector 116. The negative is released from the lumen 113 and the second connector 117 is separated from the first connector 116 during which the second connector is withdrawn from the sealing member 100.

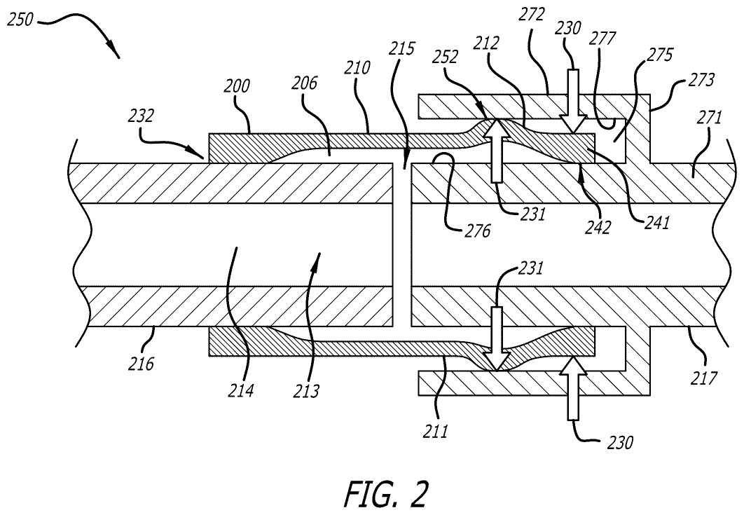

FIG. 2 illustrates a connector system 250 in a connected state. The connector system 250 generally includes a first connector 216, a complementary second connector 217, and sealing member 200. The sealing member 200 can, in certain respects, resemble components of the sealing member 100 described in connection with FIG. 1. It will be appreciated that all the illustrated embodiments may have analogous features. Accordingly, like features are designated with like reference numerals, with the leading digits increment to “2.” For instance, the annular wall is designated as “110” in FIG. 1, and an analogous annular wall is designated as “210” in FIG. 2. Relevant disclosure set forth above regarding similarly identified features thus may not be repeated hereafter. Moreover, specific features of the scaling member 100 and related components shown in FIG. 1 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the scaling member 200. Any suitable combination of the features, and variations of the same, described with respect to the sealing member 100 and components illustrated in FIG. 1 can be employed with the sealing member 200 and components of FIG. 2, and vice versa. This pattern of disclosure applies equally to further embodiments depicted in subsequent figures and described hereafter.

The sealing member 200 is sealably attached to a first connector 216 defining the fluid seal 232. The second connector 217 includes an inner annular wall 271 defining the lumen of the second connect 217. The second connector 217 further includes an outer annular wall 272 spaced radially away from the inner annular wall 271. A bottom wall 273 extends between the inner wall 271 and the outer wall 272 to define an annular cavity 275 (i.e., a receiving moat). The cavity 275 includes an inner sealing surface 276 and an outer scaling surface 277.

As shown in FIG. 2, when the connectors 216, 217 are coupled together, an extending portion 211 (i.e., an elastomeric sleeve) of the sealing member 210 is inserted within the cavity 275. When inserted, an expanded portion 212 of the sealing member 210 is disposed adjacent the outer sealing surface 277. The expanded portion 212 is sized to sealably contact the outer sealing surface 277. In other words, in the free state (i.e., absent any external forces), an outside diameter defined by the expanded portion 212 is greater than an inside diameter defined by the outer sealing surface 277. Consequently, upon coupling of the connectors 216, 217, the expanded portion 212 is compressed (i.e., forced radially inward) by the outer scaling surface 277 defining a seal 252 between the expanded portion 212 and the outer sealing surface 277 or more generally, between the sealing member 200 and the second connector 217.

In similar fashion to the sealing member 100 described above, the sealing member 200 may transition between a first engagement configuration and second engagement configuration in accordance with a change of fluid pressure 214 within the lumen 213. In the first engagement configuration, consistent with a positive/zero fluid pressure 214, the seal 252 is established between the sealing member 200 and the second connector 217. In the second engagement configuration, consistent with a negative fluid pressure 214, the seal 242 is established or enhanced between the sealing member 200 and the second connector 217.

In use, the seal 252 prevents leakage of water from the junction point 215 when a positive/zero pressure 214 is present within the lumen 213. More specifically, a positive/zero pressure 214 translates to the positive/zero chamber pressure 206 within the chamber 205 allowing the expanded portion 212 to define a contact force 231 against the outer sealing surface 277. The radially outward force 231 causes the expanded portion 212 to form the seal 252 with the outer sealing surface 277.

Similarly, air leakage into the lumen 213 is prevented by the seal 242 when a negative fluid pressure 214 is present within the lumen 213. More specifically, the negative fluid pressure 214 translates to a negative chamber pressure 206 causing the atmospheric pressure to exert a radially inward force 230 on the annular wall 210. The radially inward force causes the engagement portion 241 to form the seal 242 with the inner sealing surface 276.

A method of using the sealing member 200 may include forming the seal 252 between the sealing member 200 and the second connector 217 upon coupling of the second connector 217 with the first connector 216.

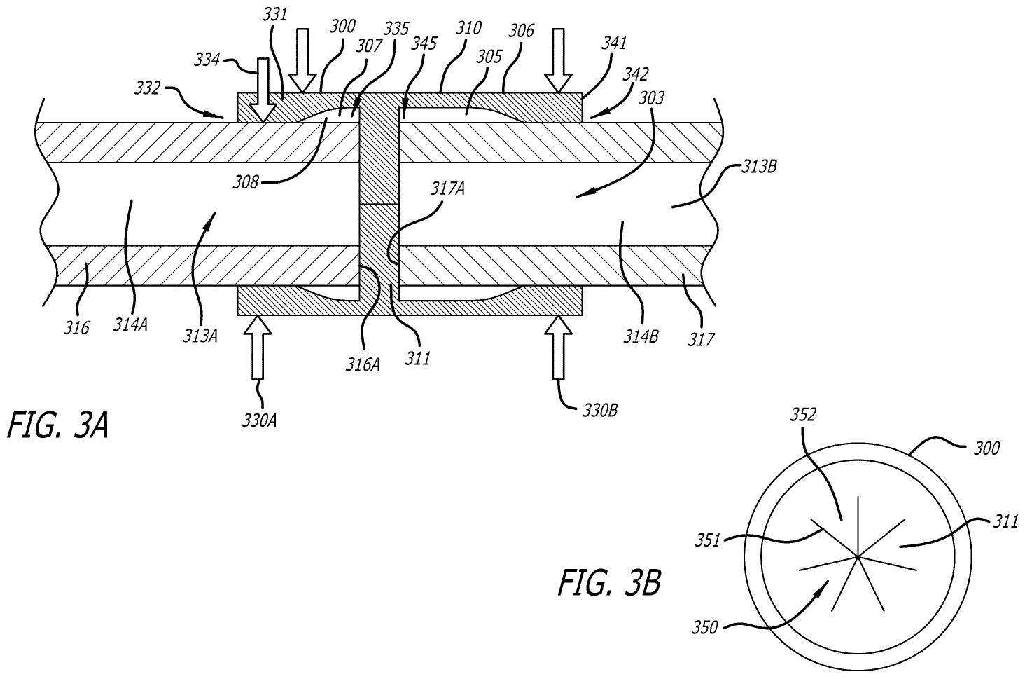

FIG. 3A illustrates a sealing member 300 in use with a first connector 316 and second connector 317. The sealing member 300 may generally define a tubular shape defining a sealing member lumen 303. The sealing member 300 includes an annular wall 310 and a septum wall 311 extending across the sealing member lumen 303. The annular wall 310 may extend circumferentially around and longitudinally along each of the connectors 316, 317. The scaling member 300 may be positioned with respect to the connectors 316, 317 so that the ends 316A, 317A of the connector 316, 317 are disposed adjacent the septum wall 311.

The sealing member 300 engages the second connector 317 via a second engagement portion 341. The second engagement portion 341 is sized to define a sliding fit between the sealing member 300 and the second connector 317. In other words, a contact force 344 between the second engagement portion 341 and second connector 317 may be sufficiently minimal to allow the second connector 317 to be inserted into and extracted from the sealing member 300.

The sealing member 300 defines an annular chamber 307 (e.g., annular space or gap) between the first connector 316 and the annular wall 310. The chamber 307 is bounded on the ends by the septum wall 311 and the first engagement portion 331. The chamber 307 may be in fluid communication with the lumen 313A via a leak path between the first connector 316 and the septum wall 311. As such, the fluid pressure 314A within the lumen 313A may define a chamber pressure 308 of the chamber 307. In an instance of a negative fluid pressure 314A within the lumen 313A, the resulting negative chamber pressure 308 causes atmospheric pressure to exert a radially inward force 330A on the annular wall 310. In such an instance, the radially inward force 330A causes an increase in the contact force 334 between the first engagement portion 331 and the first connector 316. A seal 332 between the first engagement portion 331 and the first connector 316 may be defined by the contact force 334 resulting from the negative chamber pressure 308.

Similarly, the sealing member 300 defines an annular chamber 305 (e.g., annular space or gap) between the second connector 317 and the annular wall 310. The chamber 305 is bounded on the ends by the septum wall 311 and the second engagement portion 341. The chamber 305 may be in fluid communication with the lumen 313B via a leak path between the second connector 317 and the septum wall 311. As such, the fluid pressure 314B within the lumen 313B may define a chamber pressure 306 of the chamber 305. In an instance of a negative fluid pressure 314B within the lumen 313B, the resulting negative chamber pressure 306 causes atmospheric pressure to exert a radially inward force 330B on the annular wall 310. In such an instance, the radially inward force 330B causes an increase in the contact force 344 between the second engagement portion 341 and the second connector 317. A seal 342 between the second engagement portion 341 and the second connector 317 may be defined by the contact force 344 resulting from the negative chamber pressure 306.

The sealing member 300 may be attached to the first connector 316 to inhibit or prevent longitudinal displacement of the sealing member 300 with respect to the first connector 316. In some embodiments, rotation of the sealing member 300 with respect to the first connector 316 may also be inhibited. The attachment of the sealing member 300 to the first connector 316 may define a fluid seal between sealing member 300 and the first connector 316. In other embodiments, the sealing member 300 may include a separate device (e.g., a band clamp, not shown) to define the contact force. In other embodiments, the sealing member 300 may be bonded to the first connector 316 via an adhesive. The sealing member 300 may be permanently attached to the first connector 316 or selectively attached to and/or detached from the first connector 316.

The sealing member 300 is configured to engage the second connector 317 according to a first engagement configuration and a second engagement configuration as defined by a pressure within the lumen 313B. More specifically, the sealing member 300 may be disposed in a first engagement configuration when the pressure 314B is non-negative. The sealing member 300 may transition toward the second engagement configuration in response to a pressure 314B that is negative. In the first engagement configuration, the sealing member 300 may facilitate coupling and decoupling of the connectors 316, 317. As such, longitudinal and/or rotational displacement between the sealing member 300 and the second connector 317 is allowed in the first engagement configuration.

The second engagement configuration may define enhanced engagement properties over the first engagement configuration. In some embodiments, the second engagement configuration may define a greater integrity of the seal 342 than the first engagement configuration. Similarly, the second engagement configuration may define a greater frictional force between the sealing member 300 and the second connector 317 resisting longitudinal and rotational displacement of the second connector 317 with respect to the sealing member 300.

In some embodiments, the septum wall 311 defines a face seal 335 with the end 316A of the first connector 316. The septum wall 311 may also define a face seal 345 with the end 317A of the second connector 317. As such the septum wall 311 may define a fluid seal between the connectors 316, 317.

FIG. 3B is an end view of the sealing member 300. The septum wall 311 includes one or more slits 351 extending through the septum wall 311. The slits 351 along with the corresponding elastomeric flaps 352 define a pressure actuated star valve 350. The slits 351 and flaps 352 are configured to define a septum seal in the absence of a pressure difference across the septum 311. More specifically, when the pressure difference across the septum 311 is below a defined limit, the star valve 350 is in a closed state preventing fluid flow through the sealing member 300. Conversely, when the pressure difference across the septum 311 exceeds the defined limit, the flaps 352 deflect to transition the star valve 350 to an open state allowing fluid flow through the sealing member 300.

In use, deliberate fluid flow (e.g., flow caused by a pump) through the connectors 316, 317 produces a pressure difference across the septum 311 causing the star valve 350 to open. When the fluid flow is stopped, the pressure difference is eliminated allowing the star valve 350 to close. In further use, the sealing member 300 may be attached to the first connector 316 so that when deliberate fluid flow is stopped and the connectors 316, 317 are separated, the sealing member 300 remains coupled with the first connector 316 preventing inadvertent fluid flow out of the first connector 316.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. A fluid connector system, comprising:
a first connector adjoining a second connector at a junction; and
a sealing member coupled to the first connector and the second connector around the junction, the sealing member having an annular wall comprising:
a first annular portion configured to engage the first connector;
a second annular portion configured to engage the second connector; and
an annular chamber extending between the first annular portion and the second annular portion, wherein the annular chamber is formed by the first annular portion sealing against an outer surface of the first connector and the second annular portion sealing against an outer surface of the second connector when a pressure between the sealing member and the first connector and the second connector is negative.

2. The fluid connector system according to claim 1, wherein atmospheric pressure acting on the sealing member seals the first annular portion and the second annular portion against the first connector and the second connector, respectively.

3. The fluid connector system according to claim 1, wherein the pressure between the sealing member and the first connector and the second connector is negative when a fluid pressure in a lumen through the first connector and the second connector is negative.

4. The fluid connector system according to claim 3, wherein the second annular portion is configured to slidably engage the second connector when a non-negative pressure is defined within the lumen.

5. The fluid connector system according to claim 3, wherein the second connector comprises an annular cavity enclosed at one end, the annular cavity positioned about an end portion of the second connector adjacent the junction, wherein the second annular portion is disposed in the annular cavity.

6. The fluid connector system according to claim 5, wherein a first end of the second annular portion in the annular cavity is configured to slidably engage the outer surface of the second connector, and wherein a region adjacent the first end of the second annular portion is configured to seal against a surface defining the annular cavity.

7. The fluid connector system according to claim 6, wherein the region adjacent the first end of the second annular portion is configured to seal against the surface defining the annular cavity when the pressure in the lumen is non-negative.

8. The fluid connector system according to claim 3, wherein one of the first connector or the second connector is coupled to a targeted temperature management (TTM) system.

9. The fluid connector system according to claim 8, wherein one of the first connector or the second connector is coupled to a TTM module of the TTM system.

10. The fluid connector system according to claim 8, wherein one of the first connector or the second connector is coupled to a fluid delivery line of the TTM system.

11. The fluid connector system according to claim 8, wherein one of the first connector or the second connector is coupled to a thermal contact pad of the TTM system.

12. The fluid connector system according to claim 8, wherein the pressure within the lumen is defined in accordance with operation of the TTM system.

13. The fluid connector system according to claim 3, wherein the sealing member includes a septum extending across the junction between the first connector and the second connector.

14. The fluid connector system according to claim 13, wherein the septum includes a pressure actuated valve.

15. The fluid connector system according to claim 14, wherein the pressure actuated valve is configured to prevent fluid flow through the septum when a fluid pressure across the septum is below a defined pressure limit and to permit fluid flow through the septum when the fluid pressure across the septum exceeds the defined pressure limit.

16. The fluid connector system according to claim 13, wherein the septum includes one or more slits.

17. The fluid connector system according to claim 16. wherein the one or more slits comprise a plurality of slits defining a plurality of flaps.
